(19) United States
(12) Patent Application Publication
(10) Pub. No.: US 2008/0081079 A1
(43) Pub. Date: Apr. 3, 2008

(54) Portable saline nasal spray

(76) Inventor: Byung Kwon Cha, Brooklyn Park, MN (US)

Correspondence Address: Byung Kwon Cha, Suite 4, 1401 Silver Lake Road, New Brighton, MN, 55112, US

(21) Appl. No.: 11/541,831

(22) Filed: Oct. 2, 2006

(51) Int. Cl. A61K 33/14; A61M 11/00

(52) U.S. Cl. 424/680; 128/200.23

(57) ABSTRACT

A portable saline nasal spray is a device designed to remove allergens or other harmful substances from the human nasal cavity in effective and convenient way. The portable saline nasal spray includes a spray pump that is operated by a finger, a spray nozzle that is ergonomically designed to effectively spray into the nasal cavity, and a disposable saline pouch that can be purchased separately. Not like other existing method, instead of forcing the saline solution to flow though the nasal cavity, this invention sprays the saline solution effectively over the whole surface of the nasal cavity. Because saline solution is sprayed, it requires only small amount of solution for daily use. This invention utilizes factory prepared sterile saline solution contained in disposable pouch that makes the nasal cleaning procedure much more simple and convenient. Just one small pouch of saline solution can be used for a whole week of twice a day nasal cleaning. Furthermore, because it is designed to be a leak-free portable device, it would look nice to keep anywhere, and the user can even carry it in his/her most favorite bag and use it anytime at anywhere they want.

### FIELD OF THE INVENTION

This invention relates to the field of cleaning nasal cavity with saline solution for the purpose of removing allergens or other irritants and therefore alleviating allergic rhinitis symptoms.

### BACKGROUND OF THE INVENTION

It is well known and also proved through various researches that nasal cleaning by saline solution reduces allergic symptoms such as nasal congestion and inflammation. According to many researches, 2-3 times a day of nasal wash with saline solution decreases inflammation of the nose better than other treatments, and also helps to decrease other medication use. However, in spite of all the good results from various researches, people who start nasal cleaning often quit doing it only after a few days because it is not very convenient procedure even with very new products recently came out to the market. One of recent product includes a pot and prepared salt that is packed for single use. However the direction to use it is yet not very simple. The user have to clean the pot, get clean water into the pot, put the prepared salt into the pot and stir the water to solve the salt, clean one nostril, again clean the pot and get water into the pot, put another prepared salt into the pot and stir it to solve, clean the other nostril, blow nose and clean it, clean the pot. It can be quite a messy procedure for certain people. Keeping the pot clean and neat is another matter. Also he/she may be questioning that if the tap water is safe for nasal cleaning. If the procedure is simpler, cleaner and if the saline solution is provided ready-to-use, it would be much easier to use, and more people would get the benefit from nasal wash. In this invention, the saline solution is sprayed into the nasal cavity instead of having it flow through the nasal cavity that requires a lot less amount of saline solution to use so that enables this invention to utilize factory prepared saline solution. With this invention the user can use one small pouch of saline solution, which can be purchased separately, for a whole week of twice a day nasal cleaning. Furthermore, because it is designed to be a portable device, it would look nice to keep anywhere, and the user can even carry it in his/her most favorite bag and use it anytime at anywhere they want.

Existing efforts for nasal cavity cleaning involve saline nasal flush or saline injection into nostrils where saline solution is forced to flow through the nasal cavity. It requires relatively large amount of saline solution that those existing methods have to let the user prepare the saline solution by himself/herself by mixing salt to the water. Liu in U.S. Pat. No. 6,736,792 describes a nasal-nasopharyngeal-leaning system for removing harmful substances from a human's nasal and nasopharyngeal cavities. The system includes compressible bottle, a cap for covering compressible bottle, nostril fitting, solution dispensing tube and cleaning solution. By squeezing the compressible bottle the cleaning solution is forced to flow through the nasal cavity. Erickson in U.S. Pat. No. 6,361,521 describes a system for safely irrigating nasal and sinus cavities using a portable and reusable device which can be conveniently coupled to a fluid container. Mehta in U.S. Pat. No. 6,520,384 describes an apparatus and method for preparing a pH balance saline solution and using the saline solution for rinsing a nasal passage. The apparatus includes a container having flexible sidewalls and an opening for removable cap. The side walls of the container, filled with the saline solution, are compressed to urge the saline solution though the conduit, and through the opening in the cap and into a nasal passage. As described above, there is a need still exists to make the procedure simpler and more convenient so that more people use the nasal cleaning and get benefit from it.

### SUMMARY OF THE INVENTION

In the preferred embodiment of the invention, a portable saline nasal spray is described as follows. It includes a finger operated spray pump, a spray nozzle, and replaceable disposable saline pouch. The spray nozzle is facing up, and vertically elongated to be inserted into a nostril. The tip of the spray nozzle has a narrowed and longitudinally slanted face where three small spray holes are vertically aligned along the narrow face that allows the nozzle produces upwardly angulated, vertically fanning saline spray that effectively sprays the saline solution onto the whole surface of the nasal cavity. One vertically oval shaped hole instead of vertically aligned three round holes can also be used to produce vertically fanning spray. The spray pump is located vertically at the top of the device so that the user can spray the saline solution by pressing it down with one finger of the same hand that is holing the device. A direction for daily use of the portable saline nasal spray is described in the following steps. The lid of the device is removed. The user holds the device upright with one hand in the manner that the thumb of the same hand supports the bottom of the device. The spray nozzle is inserted to one nostril until it touches the narrow opening of the nasal cavity and cannot get in any further. Spray two times by pressing down the spray pump twice with index finger or middle finger of the same hand holding the device. Remove the nozzle from the nostril at the same time the user gently sniffs in. Wash the nozzle with clean water and insert to the other nostril. Again spray two times by pressing down the spray pump twice. Remove the nozzle from the nostril at the same time the user gently sniffs in. Gently blow the nose. Repeat above procedure to spray again two times into each nostril and gently blow the nose. Wash the spray nozzle and re-place the lid of the device.

### OBJECT OF THE INVENTION

The principal object of the invention is to easily and effectively clean the nasal cavity and remove any allergen or harmful substances from it by using minimum amount of saline solution. Making the nasal cleaning device portable so that the user can use it at any time at any place they want, and be able to keep the device clean and look neat.

### BRIEF DESCRIPTION OF THE DRAWINGS

FIG. 1(a) illustrates a 3-D view of the portable saline nasal spray.

FIG. 1(b) illustrates a cross section view of the portable saline nasal spray.

FIG. 2 illustrates a cross section view of the portable saline nasal spray that is inserted into a nostril for spray.

FIGS. 3(a) and 3(b) respectively illustrate a cross section view and a 3-D view of the portable saline nasal spray with the lid on it.

FIGS. 4(a) and 4(b) respectively illustrate a cross section view and a 3-D view of the portable saline nasal spray with the lid being removed.

FIGS. 5(a) and 5(b) respectively illustrate a cross section view and a 3-D view of the portable saline nasal spray where the spraying nozzle is turned 180 degree outward and ready to spray.

FIGS. 6(a), 6(b), and 6(c) illustrate how to put disposable saline pouch into the portable saline nasal spray.

FIGS. 7(a) and 7(b) illustrate how the spray works by pressing down the spray pump.

FIGS. 8(a) and 8(b) illustrate how the portable saline nasal spray sprays the saline solution into the nasal cavity.

FIGS. 9(a), 9(b), and 9(c) illustrate a variation of the spray nozzle.

FIGS. 10(a) and 10(b) illustrate a close look of the tip of the spray nozzle and spray holes on it.

### DETAILED DESCRIPTION OF AN ENABLING AND PREFERRED EMBODIMENT

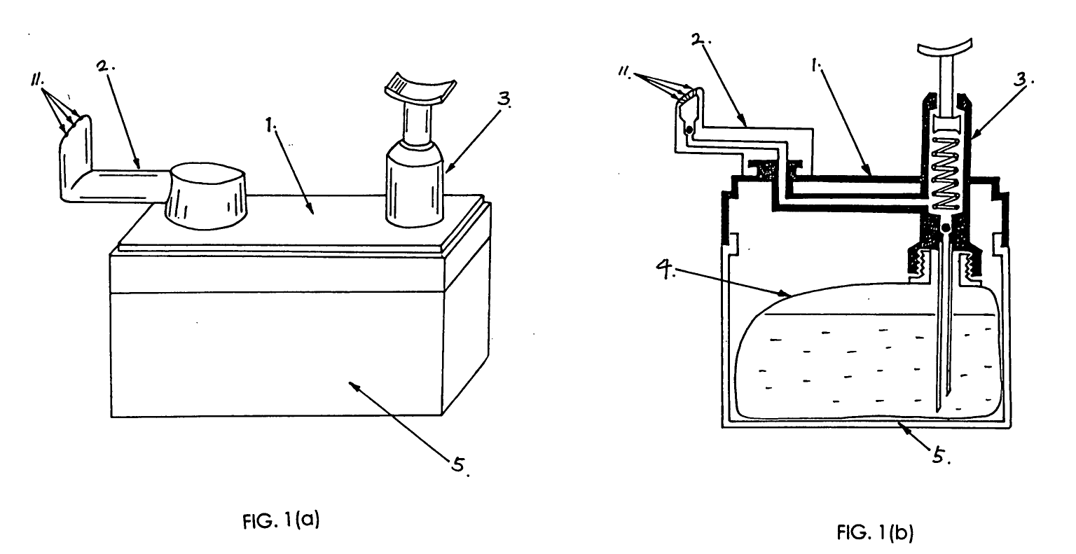

For a better understanding of the invention, we turn now to the drawings. FIGS. 1(a) and (b) offer a visual understanding of how the portable saline nasal spray appears to the eye. It has a base 1 that is the main platform where the spray pump 3 is installed, and spray nozzle 2, saline pouch 4, and pouch cover 5 are attached. The spray nozzle 2 has three small spray holes 11 aligned vertically on the slanted tip surface of the nozzle that allows the said nozzle produce upward angulated, vertically fanning saline spray so that it can effectively spray the saline solution to the whole nasal cavity. The spray nozzle 2 is attached to the base 1 like a turret that enables the spray nozzle rotates horizontally on the base 1.

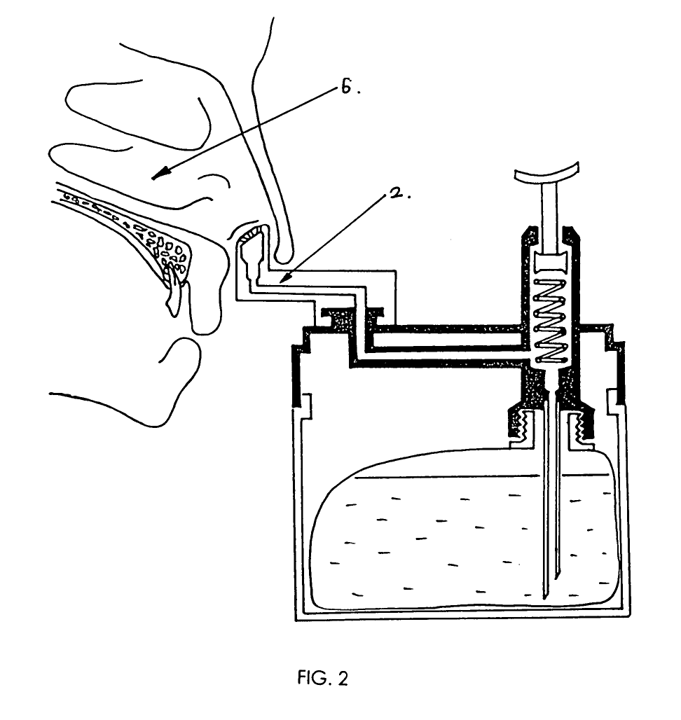

FIG. 2 shows how the spray nozzle 2 is inserted to a nostril and ready to spray. The spray nozzle is inserted deep into the nostril until the tip touches the narrow opening into the nasal cavity and is not able to get in any further. Now the portable saline nasal spray is ready to spray deep into the nasal cavity 6.

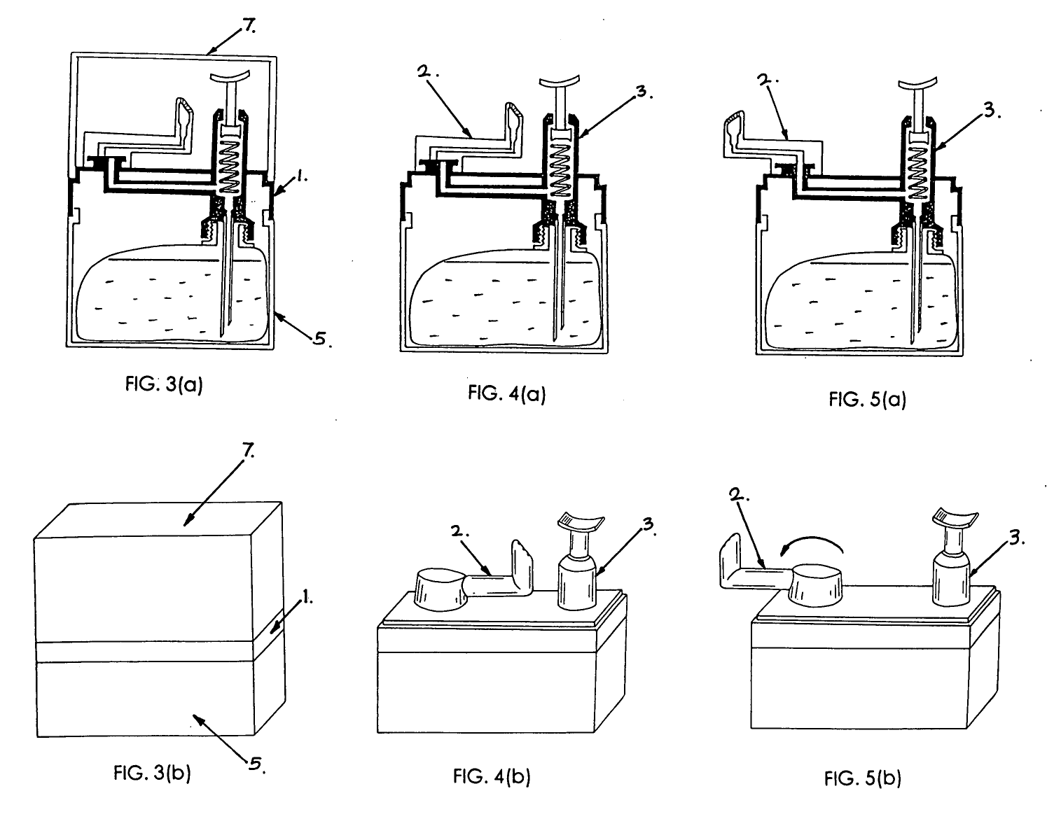

FIGS. 3(a) and 3(b) show the portable saline nasal spray with the lid 7 on it. The lid 7 protects the spray nozzle 2 and spray pump 3, keeps them clean, and makes the device look neat when it is not in use.

FIGS. 4(a) and 4(b) show the general shape of portable saline nasal spray where the lid 7 is removed. As the FIG. 4(a) illustrates, when the spray nozzle 2 is turned toward the spray pump 3 the tube between the spray pump 3 and spray nozzle 2 is blocked at the turret point that prevents leaking of saline solution during transportation.

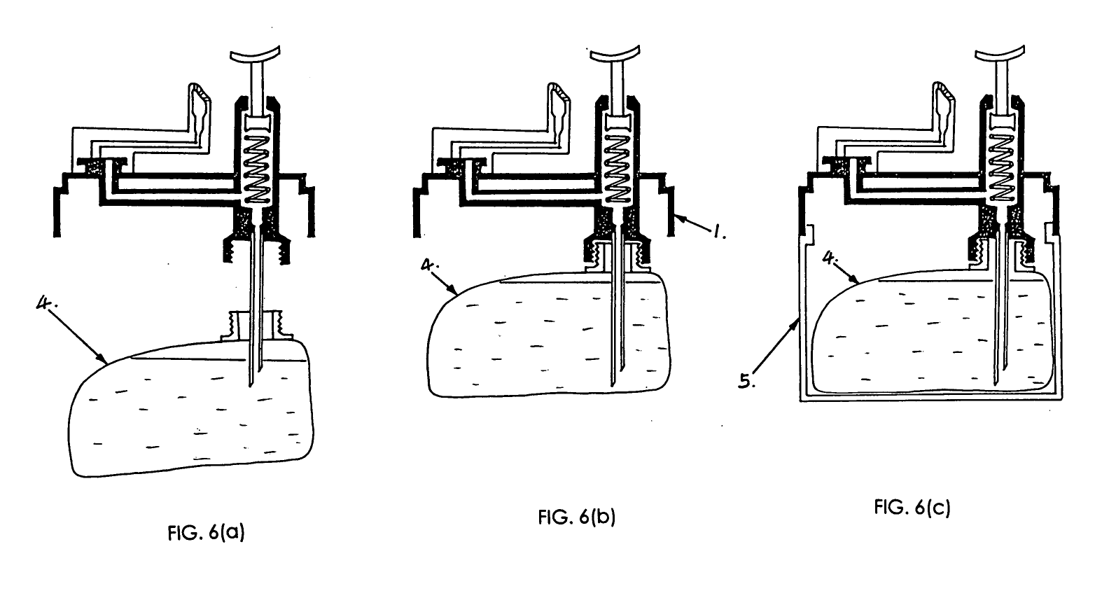

FIGS. 5(a) and 5(b) show the portable saline nasal spray that the lid 7 is removed and the spray nozzle 2 is turned outward that opens the blocked tube between the spray pump 3 and spray nozzle 2 at the turret point.

FIGS. 6(a), 6(b), and 6(c) illustrate how to put disposable saline pouch into the portable saline nasal spray. The pouch cover 5 is removed, the disposable saline pouch 4 is attached to the base 1, and the pouch cover 5 is re-placed. For this invention factory prepared sterile saline solution is applied because this invention does not require a lot of saline solution and the user can use one small pouch of saline solution for a whole week of twice a day nasal cleaning. The disposable saline pouch 4 is made of flexible plastic bag with rigid plastic bottle neck attached on it for connection to the base 1.

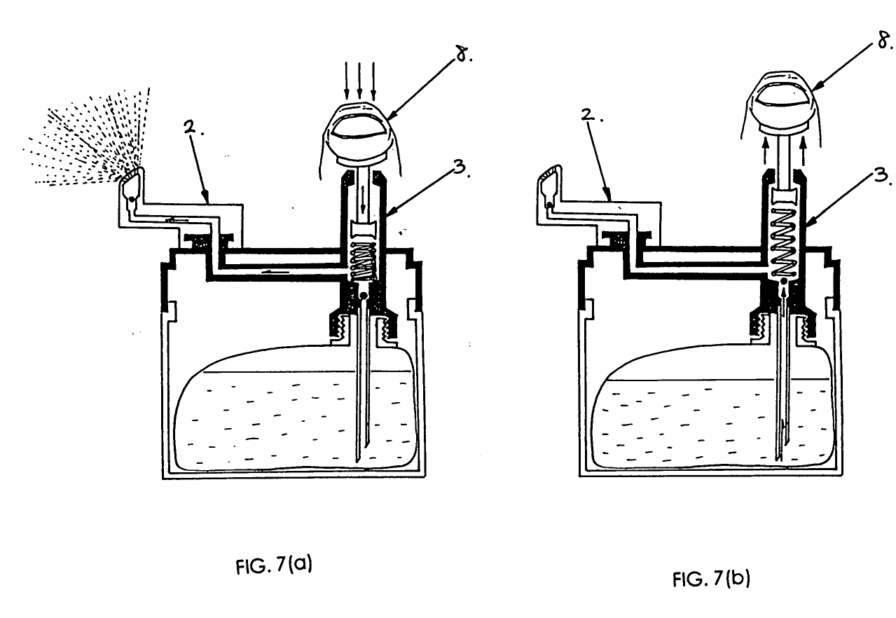

FIG. 7(a) shows the mechanism of the spray where the index finger 8 press down the spray pump 3 and the saline solution inside the pump 3 is pushed out through the tube between the pump 3 and nozzle 2 and sprays out through the spray holes. As FIG. 7(b) illustrates, when the index finger releases the spray pump 3 the saline solution from the pouch is sucked into the spray pump 3. In this procedure the saline pouch does not require air intake, instead, the bag just shrinks as the saline solution is pumped out of it that makes this device leak-free.

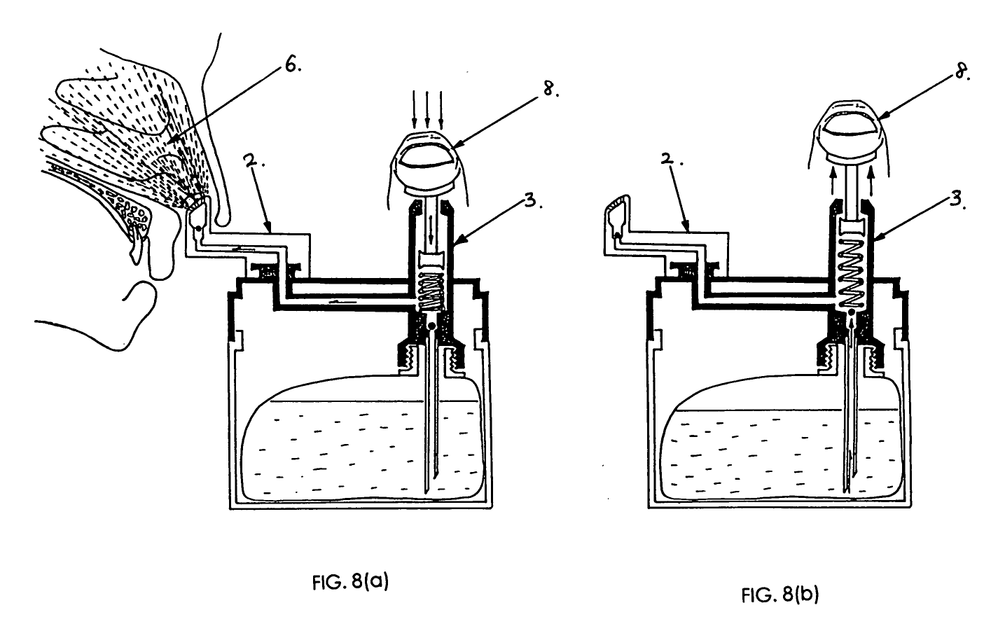

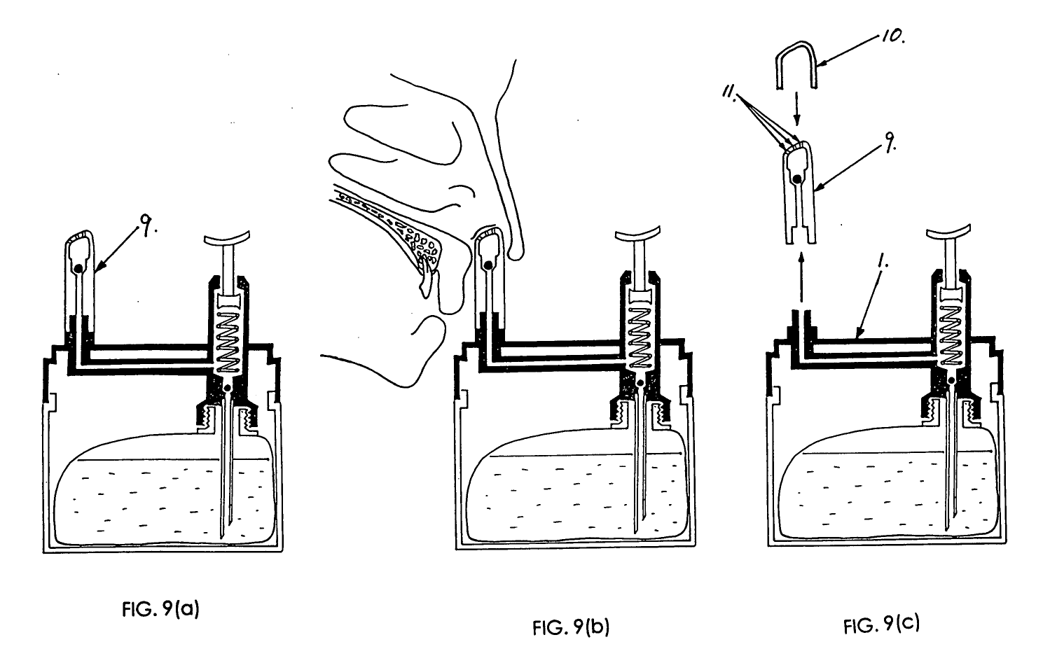

FIGS. 8(a) and 8(b) illustrate how the portable saline nasal spray sprays the saline solution into the nasal cavity 6 when the index finger 8 presses down the spray pump 3. Because the nasal cavity is vertical flat space on both side of the nasal septum, upward angulated, vertically fanning saline spray can effectively clean almost whole surface of the nasal cavity.

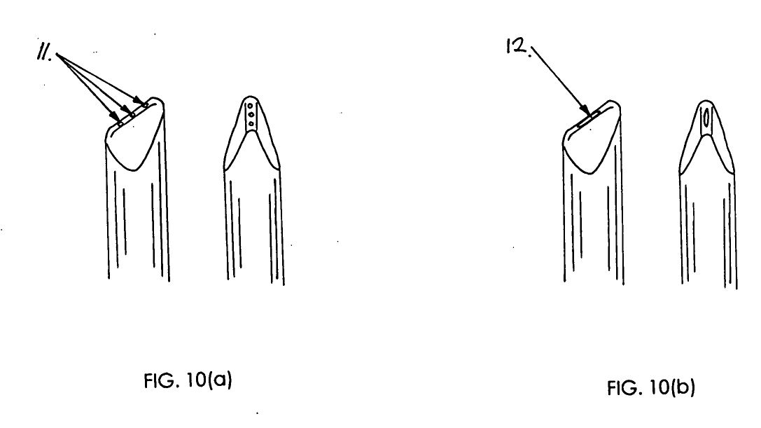

FIGS. 9(a), 9(b), and 9(c) illustrate a variation of the spray nozzle. Instead of the L-shaped spray nozzle 2 in FIGS. 1(a) and 1(b), vertically straight shape of nozzle 9 can be applied as illustrated in FIGS. 9(a), 9(b), and 9(c). In this case because there is no leak preventing stop that the spray nozzle 2 in FIG. 1 has, the spray nozzle 9 needs a nozzle cap 10 that prevents the saline solution from leaking through the spray holes 11. As it is illustrated in the FIG. 9(c), both of the spray nozzles 2 in FIG. 1 and 9 in FIG. 9 should be detachable from the base 1 for cleaning purpose.

FIGS. 10(a) and 10(b) illustrate a close look of the outer end of the spray nozzle with spray holes on it. Both type of spray nozzles (spray nozzle 2 in FIG. 1(a) and spray nozzle 9 in FIG. 9(a)) have same shape of tip at the outer end. The nozzle tip has narrowed and longitudinally slanted face where either three vertically aligned small spray holes 11 as shown in FIG. 10(a) or a vertically elongated oval spray hole 12 as shown in FIG. 10(b) in order to produce upwardly angulated, vertically fanning saline spray.

I claim:

1. A portable saline nasal spray that effectively sprays saline solution into nasal cavity for the purpose of cleaning and removing allergens or any harmful substances from nasal cavity; which consists of (a) a base that works as a main platform where the finger-operated spray pump is installed, and the lid, spray nozzle, saline pouch, and pouch cover are attached; (b) spray pump which is vertically installed on the top of said base so that the user can press it down to spray by using index finger or middle finger of the same hand holding the device; (c) faced up, and vertically elongated spray nozzle of which one end is attached to the top of said base and the other end has narrowed and longitudinally slanted face where spray holes are vertically aligned that allows the nozzle produces upwardly angulated, vertically fanning saline spray that effectively sprays the saline solution to the whole nasal cavity; (d) disposable saline pouch that contains factory prepared sterile saline solution, and has a plastic bottle neck with which said pouch is connected to the bottom of said base; (e) pouch cover that is made of rigid plastic material and attached to the bottom of said base covering said disposable saline pouch so that it protects the flexible saline pouch and also it works as a flat, stable stand of the device; (f) a lid that is made of rigid plastic material that covers whole upper part of the device when it is not in use, protecting said spray nozzle and said spray pump from being pushed or becoming dirty.

2. A portable saline nasal spray according to claim 1 wherein the saline solution is sprayed into the nasal cavity instead of being forced to flow through the nasal cavity in order to minimize the saline solution usage and maximize the cleaning effect.

3. A portable saline nasal spray according to claim 1 wherein said spray nozzle has a narrowed and longitudinally slanted face at the tip of the outer end where one or more of spray holes are located.

4. A portable saline nasal spray according to claim 1 wherein said spray hole(s) mentioned in claim 3 is either a series of small holes located along said narrowed and longitudinally slanted face, or one vertically oval shaped hole. The purpose of said spray hole(s) is producing vertically fanning saline spray.

5. A portable saline nasal spray according to claim 1 wherein said spray nozzle is detachable from the said base for cleaning purpose.

6. A portable saline nasal spray according to claim 1 wherein said spray nozzle is able to horizontally rotate at the attach point to said base. It stops the saline flow when said spray nozzle is turned to one side, and opens the flow when said spray nozzle is turned to the other side.

7. A portable saline nasal spray according to claim 1 wherein said disposable saline pouch is made of flexible plastic bag therefore it does not require air intake as the content is being emptied out.

8. A portable saline nasal spray according to claim 1 wherein said disposable saline pouch is detachable from the said base. Said disposable saline pouch has a plastic bottle neck with which said pouch is connected to the bottom of said base;

9. A portable saline nasal spray according to claim 1 wherein the saline solution is factory prepared, contained in said disposable saline pouch and can be purchased separately for saline replacement.

10. A portable saline nasal spray according to claim 1 wherein the direction for replacement of said disposable saline pouch is as follows;
remove said pouch cover from said base;
remove the empty saline pouch from the bottom of said base;
remove the cap from said bottle neck of new disposable saline pouch;
attach new saline pouch which is full of saline solution to the bottom of said base;
re-attach said pouch cover to said base.

11. A portable saline nasal spray according to claim 1 wherein the direction for use is as follows;
remove said lid from said base exposing said spray nozzle and said spray pump;
rotate said spray nozzle away from the said spray pump if it is designed to do so;
if the said spray nozzle has a nozzle cap, remove it;
hold the device up right with one hand in the manner that thumb of the same hand supports the bottom of the device, and gently insert the tip of the spray nozzle into one nostril until it touches the narrow opening of the nasal cavity and cannot move upward any further;
by using index finger or middle finger of the same hand that holds the device, press down said spray pump two times to spray onto entire surface of the nasal cavity;
remove said spray nozzle at the same time gently sniff in;
wash the tip of said spray nozzle and gently insert said tip into the other nostril until it touches the narrow part of the nostril and cannot move upward any further;
by using index finger or middle finger of the same hand that holds the device, press down said spray pump two times to spray onto entire surface of the nasal cavity;
remove said spray nozzle at the same time gently sniff in;
wash the tip of said spray nozzle;
gently blow the nose to remove excessive saline solution, mucus, and any other foreign substance from the nasal cavity;
repeat above procedures to spray again two times into each nostril and gently blow the nose;
detach said spray nozzle, wash, and re-attach it to said base;
press down said spray pump one time to clean up the nozzle;
put said nozzle cap on said nozzle tip if it has one;
put said lid back on said base of the device to protect said spray nozzle and said spray pump;
keep the device at clean and safe place.
